(19) United States
(12) Patent Application Publication
(10) Pub. No.: US 2023/0088141 A1
(43) Pub. Date: Mar. 23, 2023

(54) DEVICE FOR INHALING POWDERED ACETYLSALICYCLIC ACID AND RELATIVE USE THEREOF

(71) Applicant: EVEREST GROUP HOLDING SA, Lugano (CH)

(72) Inventor: Enrico PERFLER, Lugano (CH)

(21) Appl. No.: 17/798,883

(22) PCT Filed: Mar. 17, 2021
(86) PCT No.: PCT/IB2021/052223

§ 371 Date: Aug. 10, 2022

(30) Foreign Application Priority Data
Mar. 17, 2020 (IT) 102020000005698

(51) Int. Cl. A61M 15/00; A61M 11/00; A61K 31/616

(52) U.S. Cl. CPC A61M 15/0028; A61M 15/0001; A61M 11/002; A61K 31/616; A61M 2202/064

(57) ABSTRACT

The present invention relates to a device for delivering acetylsalicylic acid or a salt thereof for the administration thereof through oral inhalation route for the preventive or curative treatment of thrombohemolytic events, in particular for myocardial infarction.

The present invention relates to a device for delivering acetylsalicylic acid or a salt thereof for the administration thereof through oral inhalation route for the preventive and/or curative treatment of thrombohemolytic events, in particular for myocardial infarction.

Acetylsalicylic acid, known as aspirin, is an anti-platelet drug, which means that it prevents blood clotting due to inhibition of platelet function. The use of acetylsalicylic acid administered through the oral route for the preventive and curative treatment of thrombohemolytic events is known. Furthermore, the use of acetylsalicylic acid administered through the oral inhalation route which allows a rapid onset (absence of first pass through the stomach and liver) and absence of side effects of damage to the stomach (pain, bleeding and/or indigestion) potentially present in oral administration of acetylsalicylic acid is known.

Therefore, the administration of acetylsalicylic acid through the oral inhalation route is advantageous with respect to the classical oral administration using tablets is of high interest for patients with thrombohemolytic events. In particular, in the event of myocardial infarction or suspected myocardial infarction, the subject may self-administer the required dose of acetylsalicylic acid through the inhalation route, which leads to an almost immediate blood bioavailability of acetylsalicylic acid in the subject, at the onset of the infarction. As a matter of fact, the administration of active substances through the inhalation route leads said substances into the lungs and, therefore, to the absorption thereof into the bloodstream in a significantly faster way as compared to an oral gastro-enteric administration (e.g. an oral tablet).

However, the administration of powdered drugs through the inhalation route can entail problems of coughing in the subject to whom they are administered, problems of low dosages due to limited capacity of emission of the inhalers, problems of packing of the powders, drawbacks related to a just a small portion of the administered active ingredient reaching the pulmonary pathways, difficulty on the part of the subject to use inhalers, difficulty in regulating the dosage by the subject in case of multidose inhalers, problems related to bacterial sterility in case of multi-use inhalers.

Prior document WO 2016/176552 A1 discloses a composition in form of dry powder comprising a nonsteroidal anti-inflammatory drug (NSAID), such as acetylsalicylic acid. WO 2016/176552 A1 explains that said powder can be administered by means of dry-powder inhaler (DPI) or metered dose inhaler (MDI) device, comprising a mouthpiece and an actuation member to make available a dose of said NSAID to a patient through the mouthpiece. Example 9 (“Aerosol performance”) illustrates an actuation with a 60 l/min per 4 seconds flow rate. WO 2016/176552 A1 does not address drugs in the form of dry powder that can be used in inhalers that can be actuated by a subject by aspiration.

Prior document EP 2 944 343 A1 discloses a disposable inhaler, wherein a powdered formulation is contained in said inhaler in direct contact with a casing of said inhaler. The inhaler of document EP 2 944 343 A1 does not require blister capsules to contain said powdered formulation.

n order to overcome the aforementioned drawbacks, the Applicant developed a device (in short, device of the invention) comprising a single-dose inhaler, preferably a single-dose disposable inhaler, coupled with a single-dose container comprising acetylsalicylic acid or a salt thereof which is effective for the administration of acetylsalicylic acid or a salt thereof in powdered form to a subject in need through the oral inhalation route.

The device of the invention is simple to construct and cost-effective, Furthermore, the device of the invention is easy to use by any type of subject, including subjects with respiratory difficulties, for example asthmatic subjects, children and the elderly, In particular, when the device of the invention is of the disposable type, it is free from bacterial sterility-related problems. In addition, if the capsule/container comprising the active ingredient (i.e. acetylsalicylic acid) is inserted into the inhaler by the manufacturer of the device of the present invention, instead of being inserted by the user subject, the use of the device of the invention is even simpler and more immediate for the subject in need. Lastly, inhalation of the drug by means of the device of the invention is much more natural given that it is synchronised with the breathing of the patient, thus facilitating the intake of the correct dose and, therefore, the resulting relief/benefit for the subject.

These and other objects which will be clearer from the detailed description that follows, are achieved by the device, by the inhaler and by the compositions and/the mixtures of the present invention due to the technical characteristics claimed in the attached claims.

### FIGURES

FIG. 1 is an upper perspective view of the inhaler seen from the distal end thereof;

FIG. 2 is a lower perspective view of the inhaler seen from the distal end thereof;

FIG. 3 is a front perspective view of the inhaler seen from the proximal end thereof;

FIG. 4 is a top plan view of the inhaler;

FIG. 5 is a view similar to the preceding one with the cartridge fitted to the inhaler;

FIG. 6 is a cross-sectional view of the inhaler along the longitudinal centreline plane thereof, with the cartridge fitted thereon;

FIG. 7 is a view similar to the preceding one with the cartridge open for dispensing the powder comprising acetylsalicylic acid.

FIG. 8 is an upper perspective view of the air flows that pass through the inhaler when it is operating.

### DETAILED DESCRIPTION OF THE INVENTION

Forming an object of the present invention is a device for delivering acetylsalicylic acid e or a pharmaceutically acceptable salt thereof (in short, device of the present invention) comprising:
a dry powder (oral) inhaler (in short, inhaler of the present invention) having the characteristics reported below and in patent document EP 3 386 575 B1 incorporated for reference in the present description in the parts describing the inhaler (paragraphs comprised in the range from [0024] to [0041]), and
a dry powder composition for inhalation through the oral route (in short, composition of the present invention) comprising: (i) a mixture M (in short, mixture M of the present invention) comprising or, alternatively, consisting of acetylsalicylic acid or an acceptable pharmaceutical grade salt thereof; and, optionally, (ii) at least one acceptable pharmaceutical grade additive and/or excipient.

Furthermore, forming an object of the present invention is a device for delivering acetylsalicylic acid e or a pharmaceutically acceptable salt thereof (in short, device of the present invention), wherein said device can be actuated by the aspiration of a subject in need and it comprises:
a dry powder (oral) inhaler (in short, inhaler of the present invention) preferably having the characteristics reported below and in patent document EP 3 386 575 B1 incorporated for reference in the present description in the parts describing the inhaler (paragraphs comprised in the range from [0024] to [0041]), and
a dry powder composition for inhalation through the oral route (in short, composition of the present invention) comprising: (i) a mixture M (in short, mixture M of the present invention) comprising or, alternatively, consisting of acetylsalicylic acid or an acceptable pharmaceutical grade salt thereof; and, optionally, (ii) at least one acceptable pharmaceutical grade additive and/or excipient.

Said composition of the invention in the form of dry powder for inhalation through the oral route comprising acetylsalicylic acid is comprised in a cartridge (C) housed—temporarily at the time of use or permanently—in said inhaler of the present invention to obtain the device of the present invention.

Said cartridge (C) preferably comprises a cartridge wall circumscribing a containment compartment for said dry powder composition for inhalation. Said cartridge wall preferably comprises at least one moisture barrier layer, so that a moisture content of said powder is comprised in the range of from 0.01% to 15% by weight with respect to the total weight of the powder, preferably from 0.1% to 10%, more preferably from 0.5% to 5%, for a predefined period of time.

The moisture barrier layer is preferably a metal moisture barrier layer, preferably an aluminium layer. The “predefined period of time” is preferably a maximum period of time for the storage of said composition (that is to say, at least until the expiration date of said composition), under the storage conditions prescribed for said composition.

Said dry powder composition or said mixture M may comprise acetylsalicylic acid in a percentage by weight comprised in the range from 10% to 90% with respect to the total weight of the composition; preferably from 20% to 80%; more preferably from 30% to 70%.

In an embodiment, besides acetylsalicylic acid or an acceptable pharmaceutical or food grade salt thereof, said (i) mixture M further comprises a first carrier selected from the group comprising or alternatively, consisting of lactose, mannitol, a hyaluronic acid or a pharmaceutically acceptable salt thereof, preferably sodium hyaluronate, a dextran and mixtures thereof; more preferably lactose.

Preferably, the said mixture M comprises or, alternatively, consists of cetylsalicylic acid, or a salt thereof and lactose. Alternatively, said mixture M may comprise or, alternatively, consist of: acetylsalicylic acid or a salt thereof, lactose and hyaluronic acid or a salt thereof, preferably sodium hyaluronate. Said at least one first carrier, for example lactose or a mixture of lactose and a hyaluronate, may be present in the mixture M or in the composition of the invention in a percentage by weight from 1% to 60% with respect to the total weight of the mixture M or of the composition, preferably from 5% to 50%, more preferably from 10% to 40%.

In an embodiment, the acetylsalicylic acid: said at least one carrier (e.g. lactose or a lactose and a hyaluronate mixture) by weight ratio is comprised in the range from 10:1 to 1:10, preferably from 5:1 to 1:5, more preferably from 2:1 to 1:2.

Lactose (IUPAC name β-D-galactopyranosyl (1→4) D-glucopyranose, CAS 63-42-3) is a disaccharide and a dextrorotatory reducing sugar. The lactose molecule consists of a D-galactose and of a D-glucose molecule linked by a glyosidic bond (acetal) β(1-4). The aldehyde group of the glucose unit is responsible for the reducing properties of lactose. Lactose is added to the inhalation powders of the present invention to improve the efficiency at which the DPI empties after respiratory activation, the turbulence and dispersion of acetylsalicylic acid in the small airways and so as to avoid the aggregation of the powder particles to be inhaled. The lactose particles have a particle diameter such that they cannot penetrate into the deep parts of the respiratory system, hence most of the lactose settles in the oropharynx before moving on to the stomach after being swallowed.

Hyaluronic acid (for example CAS 9004-61-9) is a non-sulfated glycosaminoglycan and devoid of protein core, Hyaluronic acid and the salts thereof are macromolecules: in particular, hyaluronic acid or the salt thereof, preferably sodium hyaluronate, in the context of the present invention preferably has an average molecular weight comprised from 20 kDa to 4000 kDa, preferably comprised from 50 kDa to 1500 kDa, even more preferably comprised from 150 kDa to 1000 kDa.

In the context of the present invention, the expression hyaluronic acid salt is preferably used to indicate a salt of an alkaline or alkaline earth metal, such as for example sodium, potassium, magnesium or calcium; preferably the hyaluronic acid salt is sodium salt (sodium hyaluronate), in an embodiment, besides acetylsalicylic acid or a salt thereof, and, optionally, said first carrier selected from the group comprising or alternatively, consisting of lactose, a dextran, mannitol, hyaluronic acid or a salt thereof, and mixtures thereof, said mixture M further comprises at least one second carrier, wherein said second carrier is a stearate selected from the group comprising or, alternatively, consisting of: magnesium stearate, zinc stearate, calcium stearate, sodium stearate, lithium stearate, sodium stearyl furnarate, sodium stearoyl lactylate and the mixtures thereof; more preferably magnesium stearate. Said at least one second carrier, for example magnesium stearate may be present in the mixture M or in the composition of the invention in a percentage by weight from 0.05% to 20% with respect to the total weight of the mixture M or of the composition, preferably from 0.1% to 15%, more preferably from 1% to 10%.

Thus, the mixture M may comprise said at least one second carrier (e.g. magnesium stearate) alternatively to said first carrier or additionally to said at least one first carrier (e.g. lactose or a mixture of lactose and a hyaluronate).

When the mixture M comprises both at least one second carrier (e.g. magnesium stearate) and said at least one first carrier (e.g. lactose or a mixture of lactose and a hyaluronate), the sum of said at least one first and one second carrier may be present in the mixture M or in the composition of the invention in a percentage by weight from 1% to 60% with respect to the total weight of the mixture M or of the composition, preferably from 5% to 50%, more preferably from 10% to 40%.

In an embodiment, said (I) mixture M comprises or, alternatively, consists of: acetylsalicylic acid or a salt thereof, lactose, and a stearate, preferably magnesium stearate.

In a further embodiment, said (I) mixture M comprises or, alternatively, it consists of: acetylsalicylic acid or a salt thereof, lactose, hyaluronic acid or a salt thereof, preferably sodium hyaluronate, and a stearate, preferably magnesium stearate.

In an alternative embodiment, said (i) mixture M does not comprise a stearate,preferably it does not comprise magnesium stearate.

Said at least one acceptable pharmaceutical grade additive and/or excipient, if present in the composition of the invention together with the mixture M comprising acetylsalicylic acid and, optionally, a first carrier (e.g. lactose or a mixture of lactose and a hyaluronate) and/or a second carrier (e.g. a stearate), may be selected for example from the group comprising or, alternatively, consisting of: maltodextrin, leucine, sodium citrate, and mixtures thereof, or any other additive and/or excipient known to the man skilled in the art suitable for a composition for inhalation.

Preferably, the composition of the invention or the mixture M of the invention is in the form of dry powder suitable for inhalation through the oral route wherein said powder has an average particle diameter or a volume median geometric diameter (in short, VMGD) comprised in the range from 1 μm to 50 μm, preferably from 1 μm to 20 μm, more preferably from 1 μm to 5 μm.

The average particle diameter or the volume median geometric diameter (in short, VMGD) of the dry powder of the composition or mixture M of the present invention are measured according to standard methods known to the man skilled in the art, in particular according to technologies known in the field of powders for inhalation through the oral route. For example, the composition of the invention can be prepared through a process for the mechanical mixing of the individual components. Furthermore, or, alternatively, the composition of the invention may be prepared through a spray-drying process.

The composition of the invention or the mixture M of the invention I, comprises acetylsalicylic acid or a salt thereof, and, optionally, said at least one first carrier and/or said at least one second carrier (for example acetylsalicylic acid and lactose), may be in the form of micronized dry powder. In the context of the present invention, the expression “dry powder” is used to indicate a powder having a low moisture content, for example a powder having a content comprised in the range from 0.01% to 15% by weight with respect to the total weight of the powder, preferably from 0.1% and 10%, more preferably from 0.5% to 5%.

Forming an object of the present invention is the device for delivering acetylsalicylic acid of the present invention, comprising both the dry-powder inhaler of the present invention and the dry powder composition comprising the mixture M comprising or, alternatively, consisting of acetylsalicylic acid, for use in a method for the preventive or curative treatment of thrombohemolytic events, preferably thrombohemolytic events selected from: transient ischemic event, stroke, myocardial infarction.

Said method for the preventive or curative treatment of said thrombohemolytic events provides for the administration of acetylsalicylic acid to a subject in need through the oral inhalation route using the device of the present invention. The actuation of the device of the invention through aspiration by the subject causes the delivery of the inhalable composition of the invention in the form of dry powder by the inhaler of the invention and thus the administration of a sufficient curative dose of acetylsalicylic acid to said subject through the inhalation route.

The method of treatment of the present invention or the device of the present invention for use in the method for the preventive or curative treatment of a thrombohemolytic event provides for the administration of a low amount of acetylsalicylic acid—lower than the dose of oral acetylsalicylic acid (for example, lower than 81 mg, such as “baby aspirin” or “small-dose aspirin”)—by inhalation. The dose of acetylsalicylic acid (or relative salt) administered through the inhalation route by means of the device of the present invention may be lower than about 40 mg and it may vary from about 1 mg to about 40 mg, from about 4 mg to about 25 mg, from about 6 mg to about 20 mg, from about 8 mg to about 15 mg, from about 10 mg to about 13 mg, or about 1 mg, about 2 mg, about 3 mg, about 4 mg, about 5 mg, about 6 mg, about 7 mg, about 8 mg, about 9 mg, about 10 mg, about 11 mg, about 12 mg about 13 mg, about 14 mg, about 15 mg, about 16 mg, about 17 mg, about 18 mg, about 19 mg or about 20 mg of acetylsalicylic acid, Alternatively, the dose of acetylsalicylic acid administered through the inhalation route by means of the device of the present invention may be lower than about 80 mg or lower than about 100 mg, da circa 1 mg to about 75 mg, from about 2 mg to about 60 mg, from about 5 mg to about 40 mg, from about 10 mg to about 30 mg, from about 12 mg to about 25 mg, from about 15 mg to about 20 mg, from about 60 mg to about 95 mg, from about 50 mg to about 100 mg, from about 50 mg to about 80 mg, from about 40 mg to about 80 mg, from about 20 mg to about 30 mg, from about 30 mg to about 40 rng, from about 40 mg to about 50 mg, from about 50 mg to about 60 mg, from about 60 mg to about 70 mg, from about 70 mg to about 80 mg, from about 80 mg to about 90 mg or from about 90 mg to about 100 mg.

The single-dose dry powder inhaler of the present invention (inhaler described in EP 3 386 575 B1), preferably single-dose disposable inhaler, essentially consists of a pipe-shaped body with a first portion (in short, housing portion) suitable for housing a self-piercing cartridge containing the medicinal product (or active substance or composition of the invention), connected—through a grid—to a second portion, for dispensing the powder, which the patient puts in the mouth.

Preferably, said first portion 1 and said second portion 2 consist of the same polymeric material in a single piece.

Said polymeric material is preferably selected from polyethylene (PE), low-density polyethylene (LDPE), medium-density polyethylene (MDPE), high-density polyethylene (HDPE), polypropylene (PP), polyvinyl acetate (PVC) and polycarbonate (PC).

Said second portion for dispensing the powder (in short, dispensing portion) is provided with a lower duct which allows to obtain a secondary air flow which supports and directs the primary flow which conveys the powdered medicinal product, so that the primary flow can go past the tongue of the patient. This allows to prevent the excessive deposit of the medicinal product in the oropharyngeal cavity and it results in greater treatment efficacy due to the increased amount of medicinal product reaching the lungs. A further advantage deriving from the presence of the secondary flow lies in the greater separation of the active substance from the carrier (e.g. lactose, mannose, etc.) due to the secondary flow which impacts and directs the primary flow. Furthermore, the inhaler of the invention is suitable to house a self-piercing cartridge, thus, there is no need for the presence of other elements for piercing the cartridge.

The main characteristic of dry-powder inhalers, such as the inhaler of the present invention, lies in that they are activated directly by inspiration by the patient and in that the drug (i.e. acetylsalicylic acid) is inhaled simultaneously with the breathing of the patient. Therefore, the key to a perfect performance of the device lies in the design of the inhaler, which must guarantee a minimum resistance in the dispensing flow and a formation of turbulence, which ensure a good separation of the drug from the carrier and/or excipient.

In other words, the delivery device subject of the present invention has no actuation members which artificially generate (for example by means of a pump or blower) an air flow make said composition available to the subject.

In the case of single-dose inhalers described in the prior art and different from the inhaler of the present invention, it was observed that in use by patients with asthma or poor respiratory capacity, the performance of the device is not optimal in terms of both dispersion of inhalable drug particles and in terms of formation of resistance within the flow. A further drawback of the single-dose inhalers described in the prior art and different from the inhaler of the present invention lies in the difficulty, especially for elderly patients, to correctly position and lock the self-piercing cartridge on the housing portion of the inhaler, so that there are no problems when opening the cartridge and all the drug falls to the expected point.

Still another drawback observed in single-dose inhalers described in the prior art and different from the inhaler of the present invention lies in the fact that, in said inhalers, in the dispensing duct the septum which partitions the lower duct from the upper duct, where the primary flow carrying the powder passes—extends up to the end of the delivery duct which is placed in the mouth of the patient. As a result, the tongue of the patient could occlude the outlet of the lower duct, thus preventing secondary flow from being dispensed when the patient inspires to inhale the drug.

The single-dose dry powder inhaler of the present invention, preferably single-dose disposable inhaler, is free from the aforementioned drawbacks.

The single-dose dry powder inhaler of the present invention is similar to the one described in EP 1 799 289, but provided with a support base for the self-piercing cartridge, obtained inside the housing portion. In said support base there are obtained oriented flow channels extending between at least three air intakes and the powder-fall region on the bottom of the housing portion. Other preferred characteristics of the inhaler of the present invention relate to the shortening of the septum which partitions the lower duct from the upper duct so that it does not extend up to the end of the dispensing duct, as well as the creation of multiple separate air inlet regions for supplying the lower duct through an air intake obtained at the distal end of the latter.

A first significant advantage of the inhaler of the present invention lies in the presence of oriented flow channels which during inhalation by the patient, even under reduced-aspiration conditions help the formation of vortices which intersect in the medicinal powder-fall region creating turbulence such to determine a very strong flow rate which facilitates the dispersion of the inhalable drug particles, thus increasing the percentage of inhalability of the drug.

A second significant advantage of the inhaler of the present invention lies in the ease of fitting of the self-piercing cartridge and the great stability of the latter provided by the support base, so that the cartridge can be pre-fitted on the inhaler at the factory (single-dose disposable inhaler), so that even patients with difficulty in handling and opening the cartridge do not risk scattering the medicinal powder out of the intended fall region.

A further advantage of the, inhaler of the present invention derives from the fact that the, shortened partitioning septum of the dispensing duct ensures that the secondary flow is always dispensed so that it begins its action of supporting the primary flow even before reaching the end of the dispensing duct located in the mouth of the patient. Further, the multiple regions for the inlet of air to the lower duct ensure a secondary flow with an adequate flow rate and minimum resistance for an easy flow of the air in the lower duct

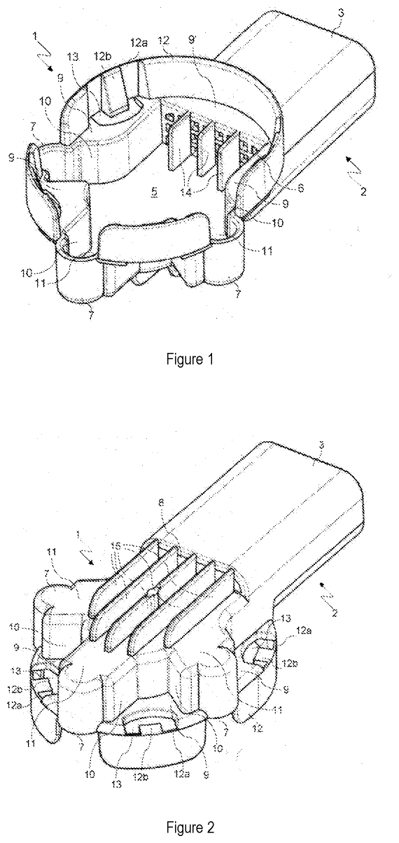

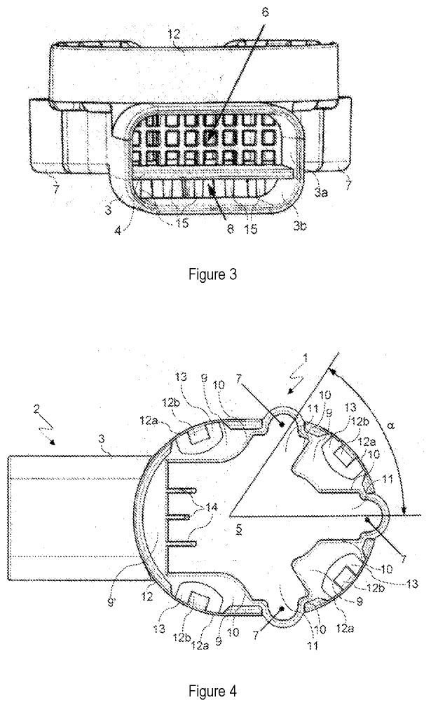

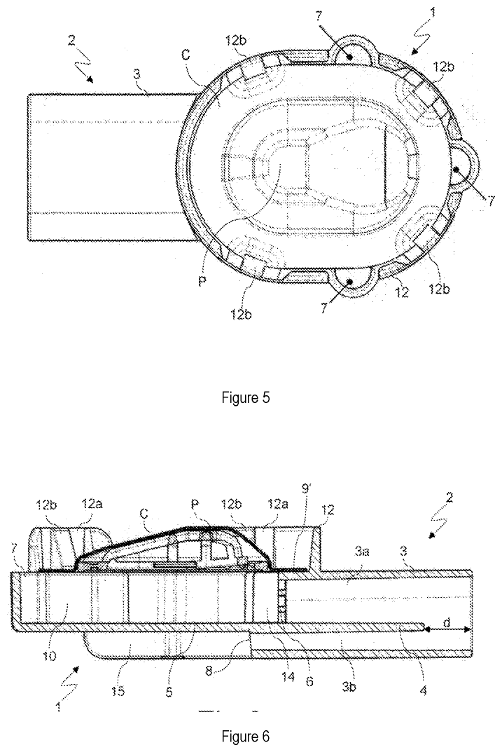

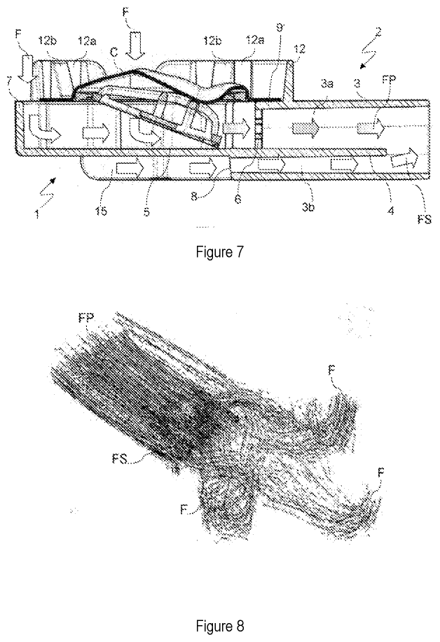

With reference to FIGS. 1 to 8, it is observable that a single-dose inhaler according to the present invention, preferably a single-dose disposable inhaler, conventionally consists of a substantially pipe-shaped hollow body having a first portion 1, for housing a cartridge C o a powdered medicinal product (FIGS. 5-7), and a second portion 2, for dispensing the medicinal through a dispensing duct 3, whose end is suitable to be placed in the mouth of the patient. Such duct 3 is horizontally partitioned—by a partitioning septum 4—into an upper duct 3a, which dispenses the primary flow FP which conveys the powder (indicated by the shaded arrows in FIG. 7), and a lower duct 3b which dispenses the powder-free secondary flow FS (Indicated by the white arrows in FIG. 7), which supports and directs the primary flow FP at the time of inhalation.

The connection between the powder-fall region 5, on the bottom of the housing portion 1, and the upper duct 3a is obtained by means of a grid 6 dimensioned so as to prevent the through-flow of pieces of the cartridge that could fall therefrom at the time of delivery of the powder. Furthermore, the grid 6 also has the purpose of ensuring, facilitated by the formation of turbulence, a relatively uniform spread of the particles towards the airways of the user and of facilitating the separation and dispersion of possible agglomerates of powder, also separating the drug particles from those of the excipient so that they can better reach the lungs.

Preferably, the grid 6 consists of a single piece made of the same polymeric material as the first portion 1 and the second portion 2, said polymeric material being more preferably selected from polyethylene (PE), low-density polyethylene (LDPE), medium-density polyethylene (MDPE), high-density polyethylene (HDPE), polypropylene (PP), polyvinyl acetate (PVC) and polycarbonate (PC).

The aspiration of the air forming the primary flow FP for conveying the powder occurs through at least three air intakes 7 which act as inlets to said powder-fall region 5; preferably, a central air intake cantered on the longitudinal centreline plane of the inhaler, in a distal position, and two other lateral air inlets arranged symmetrically with respect to said centreline plane. The aspiration of the air forming the support secondary flow FS occurs through an air intake 8 formed at the distal end of the lower duct 3b.

The air flows F entering through the three air intakes 7 combine to form the primary flow FP which conveys the powder from the region 5, through the grid 6, along the upper duct 3a to the mouth of the patient, with the support of the secondary flow FS coming from the lower duct 3b at the moment of entry into the mouth.

A first innovative aspect of the inhaler according to the invention lies in the presence of a support base for supporting the cartridge C, said base being obtained inside the housing portion 1 by means of a plurality of horizontal support surfaces 9 which are preferably obtained on a plane corresponding to the top part of the upper duct 3a, as shown in the sections of FIGS. 6 and 7. The vertical walls 10 which connect said horizontal surfaces 9 with the powder-fall region 5, on the bottom of the housing portion 1, define three flow channels 11 extending between the three air intakes 7 and said region 5.

More specifically, the longitudinal axes of the two channels 11 extending from the lateral air intakes 7 form an angle a with the centreline plane (FIG. 4) which is approximately equal to 55° but may vary by ±20%. With this orientation of the channels 11, the air flows F coming from the three air intakes 7 converge substantially at the point of the region 5 where most of the medicinal powder falls, that is at the position where the region P, on which the patient applies pressure using the finger to open the cartridge C (FIGS. 5-7) is located. This allows to obtain an adequate turbulence, as shown in FIG. 8, sufficient to obtain the dispersion of the inhalable drug particles even under reduced-aspiration conditions.

It should be observed that should the air intakes 7 be more in number, for example four or five, they will still be obtained preferably symmetrical with respect to the centreline plane and in a position such that the aforementioned region P is comprised between the intersections of the axes of the channels 11. Furthermore, although the walls 10 of the channels 11 have been shown parallel to each other, they could also be divergent or convergent so as to adjust and further direct the flow F of the air flowing in from the air intakes 7, which preferably have a passage section measuring at least 6.4 mm².

The perimeter wall 12 of the housing portion 1, extending in the upper part of the latter above the support base, is provided with coupling means so to obtain an easy and effective locking of the cartridge C onto the inhaler. More specifically, at each support surface 9, the wall 12 has a region 12a with reduced thickness which makes it elastically flexible and at the centre of each region 12a there is a triangular tooth 12b which projects thereinto. Such tooth 12b is obtained with an inclined surface extending from the top part of the wall 12 toward the support base, and it ends below with a horizontal base so as to form a chute for the insertion of the cartridge C from above and an undercut for the locking thereof on the support base (FIGS. 5-7).

It should be observed that in order to ensure the required flexibility of the regions 12a carrying the locking teeth 12b, the support surfaces 9 are obtained spaced from the regions 12a, thus leaving an empty space 13 around each tooth 12b. Furthermore, the base of the teeth 12b is located slightly higher than the plane of the support base so as to take into account the thickness of the perimeter edge of the cartridge C which engages the teeth 12b.

In an embodiment, the cartridge C is positioned on the inhaler by means of a fully automated production line. A defined pressure—which causes the four elastic regions 12a to be pushed outwards and then return to the initial position thereof when locking the cartridge C on the inhaler using the four teeth 12b which engage the edge of cartridge C—is applied on the perimeter edge of the cartridge C. In this manner, during the automated production step, each single cartridge C, after being filled and sealed, is assembled with the inhaler thus obtaining a disposable (single-dose) for inhaling powdered medicinal products that is easy to use for anyone.

The wall 12 also encloses a distal portion 9′ of the top part of the upper duct 3a, such distal portion 9′ thus being part of the support base of the cartridge C. Likewise, three vertical septa 14—extending over the entire height of the grid 6 and divide it into four air inlet regions to split and align the primary flow FP flowing in from the region 5—are part thereof, said septa 14 being preferably arranged so as to obtain inlet regions of the same width.

Similarly, in a second innovative aspect of the present inhaler, the air intake 8 of the lower duct 3b is partitioned into a plurality of air inlet regions, still preferably of the same width, by a plurality of elongated septa 15 (five in the illustrated example but they could be from one to seven) extending below the powder-fall region 5, so as to facilitate the flow of the secondary flow FS, preventing the onset of any resistance. Moreover, these elongated septa 15 also have an ergonomic function in that they prevent the fingers of the patient holding the inhaler from closing, even only partially, the air intake 8 thus reducing the flow rate and the effectiveness of the secondary flow FS.

As mentioned above, the secondary flow FS supports the primary flow FP and prevents powder, even due to gravity, from being deposited on the tongue of the patient or on other walls of the oropharyngeal cavity. Such support and directing function is particularly important if the patient holds the distal end of the inhaler tilted too much upwards.

This support effect, same case applying to the effect of separating the drug from the excipient, can be increased or decreased by varying the sections of the air intakes 7, 8 and/or the ratio between the inlet sections of the ducts 3a, 3b (intended as the actual throw-flow sections net of the grid 6 and the septa 15). More specifically, the minimum inlet section of the upper duct 3a is 25.6 mm²±20% and the minimum inlet section of the lower duct 3b is 14.3 mm²±20%, and the ratio between said sections may vary in the range from 1:1 to 9:1.

In other words, the two flows FP, FS may be modulated by designing the various portions of the inhaler in order to obtain various embodiments with different inhalation characteristics for different specific applications. Among the design parameters, in a third innovative aspect of the present inhaler, there is also the distance d (FIG. 6) between the proximal end of the dispensing duct 3 and the proximal end of the partitioning septum 4, such distance d being preferably comprised between 4 and 7 mm.

Therefore, it is clear that the embodiment of the inhaler according to the invention described and illustrated above represents only one example susceptible of numerous variations. In particular, although the portions 1, 2 have been illustrated connected at 90°, it is clear that the monolithic body of the inhaler may also be obtained even with a smaller angle between said portions, for example 45° or 60°.

Furthermore, the number, shape and arrangement of the air intakes 7 may be freely varied and therefore the air intakes 7 could be entirely different from the projecting semi-circular shapes illustrated above. For example, the air intakes 7 could be simple openings of any shape obtained in the lower wall of the housing portion 1 below the support base.

Preferred embodiments (Ens) of the present delivery device are illustrated below:

E1. A device for delivering acetylsalicylic acid or a salt thereof actuatable by the aspiration of a subject in need comprising:
a dry powder composition for inhalation comprising
(i) a mixture (M) comprising or, alternatively, consisting of acetylsalicylic acid or an acceptable pharmaceutical grade salt thereof; and, optionally,
(ii) at least one acceptable pharmaceutical grade additive and/or excipient; and
a dry-powder inhaler comprising a substantially pipe-shaped hollow body comprising a first portion (1) for housing a cartridge (C) comprising said dry powder composition for inhalation, and a second portion (2) connected to said first portion (1) for dispensing the powder composition by means of a primary air flow (FP) carrying the powder from an inner fall region (5), located at the bottom of said first portion (1), along a dispensing duct (3) whose end is suitable to be placed in the mouth of the subject, said dispensing duct (3) being horizontally partitioned—by a partitioning septum (4)—into an upper duct (3a) which dispenses said primary air flow (FP) and a lower duct (3b) which dispenses a powder-free secondary air flow (FS), the aspiration of the air forming the primary flow (FP) occurring through at least three air intakes (7) formed in the first portion (1) which are preferably arranged symmetrically with respect to the longitudinal centreline plane of the inhaler, the aspiration of the air forming the secondary flow (FS) occurring through an air intake (8) obtained at the distal end of said lower duct (3b),

the inhaler being characterised in that said base for supporting the cartridge (C) includes a plurality of horizontal support surfaces (9) projecting into the first portion (1) and oriented flow channels (11) formed in the support base which extend between said at least three air intakes (7) and inner powder fall region (5).

E2. The delivery device according to El, wherein said first portion (1) and said second portion (2) consist of the same polymeric material made of a single piece, said polymeric material being preferably selected from polyethylene (PE), low-density polyethylene (LDPE), medium-density polyethylene (MDPE), high-density polyethylene (HDPE), polypropylene (PP), polyvinyl acetate (PVC) and polycarbonate (PC).

E3. The delivery device according to E2, wherein, in said dry-powder inhaler, a connection between the inner region (5) for the fall of the powder and the upper duct (3a) is obtained by means of a grid (6), and wherein said grid (6) consists of a single piece made of the same polymeric material as the first portion (1) and the second portion (2).

E4. The delivery device according to any one of E1-E3, wherein said cartridge (C) comprises a cartridge wall circumscribing a compartment for containing said dry powder composition for inhalation wherein said cartridge wall comprises at least one moisture barrier layer, preferably a metal moisture barrier layer, so that a moisture content of said powder is comprised in the range of from 0.01% to 15% by weight with respect to the total weight of the powder, preferably from 0.1% to 10%, more preferably from 0.5% to 5%, for a predefined period of time.

E5. The delivery device according to any one of E1-E4, wherein said mixture (M) comprises or, alternatively, consists of acetylsalicylic acid or a salt thereof and at least one first carrier, wherein said at least one first carrier is selected from the group comprising or, alternatively, consisting of: lactose, mannose, a hyaluronic acid or a pharmaceutically acceptable salt thereof, preferably sodium hyaluronate, a dextran, and mixtures thereof.

E6. The delivery device according to any one of E1-E5, wherein said powder composition or said mixture (M) has a volume median geometric diameter (VMGD) comprised in the range from 1 μm to 50 μm, preferably from 1 μm to 20 μm, more preferably from 1 μm to 5 μm.

E7. The delivery device according to any one of E1-E6, wherein said dry powder composition comprises acetylsalicylic acid or a salt thereof in a percentage by weight comprised in the range from 10% to 90% with respect to the total weight of the composition; preferably from 20% to 80%; more preferably from 30% to 70%.

E8. The delivery device according to any one of E1-E7, wherein said inhaler is characterised in that the partitioning septum (4) is shorter than the dispensing duct (3), preferably by a distance (d) comprised between 4 and 7 mm.

E9. The delivery device according to any one of E1-E8, wherein said inhaler is characterised in that the air intake (8) of the lower duct (3b) is partitioned into a plurality of air inlet regions, preferably having the same width, by one or more elongated septa (15), preferably not more than seven, extending below the powder fall region (5).

E10. The delivery device according to any one of E1-E9, wherein said inhaler is characterised in that the first portion (1) comprises a perimeter wall (12) extending above the support base and it is provided with coupling means for locking the cartridge (C) onto the delivery device.

E11. The delivery device according to any one of E1-E10, wherein said inhaler is a single-dose disposable inhaler; preferably wherein the cartridge (C) comprising the dry powder composition is fixed in the inhaler support base.

E12. The delivery device according to any one of E1-E11, wherein said dry powder composition for inhalation is a composition for use in the curative or preventive treatment of thrombohemolytic events.

E13. The delivery device according to any one of the preceding Ens, wherein said dry powder composition for inhalation is a composition for use in the curative or preventive treatment of thrombohemolytic events selected from: transient ischemic event, stroke, myocardial infarction.

Embodiments (FRns) of the single-dose inhaler of the present invention are as follows:

FR1. Powdered medicinal product inhaler consisting of a substantially pipe-shaped hollow body having a first portion (1), for housing a powdered medicinal product cartridge (C), and a second portion (2) connected substantially perpendicularly to said first portion (1) for dispensing the medicinal product by means of a primary air flow (FP) carrying the powder from an inner fall region (5), located at the bottom of said first portion (1), along a dispensing duct (3) whose end is suitable to be placed in the mouth of the patient , said dispensing duct (3) being horizontally partitioned—by a partitioning septum (4)—into an upper duct (3a) which dispenses the primary air flow (FP) and a lower duct (3b) which dispenses a powder-free secondary air flow (FS), the aspiration of the air forming the primary air flow (FP) being obtained by means of at least three air intakes (7) formed in the first portion (1) which are preferably arranged symmetrically with respect to the longitudinal centreline plane of the inhaler, the aspiration of the air forming the secondary flow (FS) being obtained by means of an air intake (8) obtained at the distal end of said lower duct (3b), the inhaler being characterised in that said base for supporting the cartridge (C) includes a plurality of horizontal support surfaces (9) projecting into the first portion (1), oriented flow channels (11) formed in the support base extending between said at least three air intakes (7) and the powder-fall inner region (5).

FR2. Inhaler according to FR1, characterised in that the longitudinal axes of the channels (11) extending from side air intakes (7) form a 55°±20% angle (α) with the centreline plane.

FR3. Inhaler according to one of the preceding FR1-FR2, characterised in that said horizontal support surfaces (9) projecting into the first portion (1) are formed on a plane corresponding to the top part of the upper duct (3a) so that a distal portion (9′) of the latter is also part of the support base.

FR4. Inhaler according to any one of the preceding FR1-FR3, characterised in that the connection between the internal powder fall region (5) and the upper duct (3a) is obtained by means of a grid (6).

FR5. Inhaler according to one of the preceding FR1-FR4, characterised in that it further comprises three vertical septa (14) extending over the entire height of the grid (6) and partition it into four air inlet regions, said vertical septa (14) being preferably arranged so that said inlet regions have the same width.

FR6. Inhaler according to one of the preceding FR1-FR5, characterised in that the partitioning septum (4) is shorter than the dispensing duct (3), preferably by a distance (d) comprised between 4 and 7 mm.

FR7. Inhaler according to one of the preceding FR1-FR6, characterised in that the first portion (1) comprises a perimeter wall (12) extending above the support base and it is provided with coupling means for locking the cartridge (C) onto the inhaler.

FR8. Inhaler according to one of the preceding FR1-FR7, characterised in that, at each support surface (9), the perimeter wall (12) has an elastic region (12a) with reduced thickness having a central triangular tooth (12b) projecting thereinto, said tooth (12b) being obtained with an inclined surface extending from the top part of the wall (12) toward the support base and with a lower horizontal base so as to form a chute for the insertion of the cartridge (C) from above and an undercut for the locking thereof onto the base of support.

FR9. Inhaler according to one of the preceding FR1-FR8, characterised in that the air intake (8) of the lower duct (3b) is partitioned into a plurality of air inlet regions, preferably having the same width, by one or more elongated septa (15), preferably not more than seven, extending below the powder fall region (5).

FR10. Inhaler according to one of the preceding FR1-FR9, characterised in that the minimum inlet section of the upper duct (3a) is 25.6 mm²±20%, the minimum inlet section of the lower duct (3b) is 14.3 mm²±20%, and the ratio between said sections may vary in the range from 1:1 to 9:1.

FR11. Inhaler according to one of the preceding FR1-FR10, characterised in that the air intakes (7) for the primary air flow (FP) consist of projections, preferably semi-circular, obtained in the lower wall of the first portion (1) below the support base.

FR12. Disposable single dose device for the inhalation of powdered medicinal products, characterised in that it comprises an inhaler according to one of FR9-FR11 and a self-piercing cartridge comprising the dry powder composition of the invention locked on the support base of said inhaler, said cartridge (C) having a symmetrical longitudinal plan shape substantially corresponding to the shape of said support base and a region (P) designed to receive the pressure of a patient's fingers to open the cartridge (C), said region (P) being aligned to the longitudinal symmetry plane of the cartridge (C).

The administration of the composition of the invention in the form of dry powder for inhalation by means of the delivery device of the invention, actuated by the aspiration of the subject in need, is such to make effective the administration of the effective dose and to maximise the intrinsic effectiveness of the compositions of the invention itself.

Furthermore, when inhaled using the inhaler of the invention actuated by means of a single aspiration action by the subject to whom the composition is administered, the composition of the invention in the form of dry powder for inhalation creates a good inhalable fraction and an accurate therapeutically active dose of the active ingredient.

The expression “inhalable fraction” is used to indicate an index of the particle percentage of active ingredient which reaches the lungs (deep area) in a subject. The inhalable fraction, also referred to as the fine particle fraction (FPF), is evaluated using appropriate in vitro apparatus such as Multistage Cascade Impactor or Multi Stage Liquid Impinger (MLSI) according to the procedures indicated in common pharmacopeia. FPF is calculated from the ratio between the dispensed dose and the fine particle mass (of fine particle dose, in short FPD). An inhalable fraction greater than 30% is an index of good inhalation performance.

The expression “accurate therapeutically active dose of the active ingredient” refers to a composition in which the variation between the average dispensed daily intake and the average emitted dose is equal to or lower than 15%, preferably lower than 10%.

Unless specified otherwise, the expression composition or mixture or other comprising a component at an amount “comprised in a range from x to y” is used to indicate that said component can be present in the composition or mixture or other at all the amounts present in said range, even though not specified, extremes of the range comprised.

Unless specified otherwise, the indication that a composition or mixture “comprises” one or more components or substances means that other components or substances can be present besides the one, or the ones, indicated specifically.

In the context of the present invention, the expression “treatment method” is used to indicate an intervention on a subject in need, comprising the administration of a composition or mixture of substances with the aim of eliminating, reducing/decreasing or preventing a disease or ailment and symptoms or disorders thereof.

In the context of the present invention, the expression “subject/s” is used to indicate human subjects.

1. A device for delivering acetylsalicylic add or a salt thereof actuatable by the aspiration of a subject in need comprising:
a dry powder composition for inhalation comprising
(i) a mixture (M) comprising or, alternatively, consisting of acetylsalicylic add or an acceptable pharmaceutical grade salt thereof; and, optionally,
(ii) at least one acceptable pharmaceutical grade additive and/or excipient; and
a dry-powder inhaler comprising a substantially pipe-shaped hollow body comprising a first portion (1) for housing a cartridge (C) comprising said dry powder composition for inhalation, and a second portion (2) connected to said first portion (1) for dispensing the powder composition by means of a primary air flow (FP) carrying the powder from an inner fall region (5), located at the bottom of said first portion (1), along a dispensing duct (3) whose end is suitable to be placed in the mouth of the subject, said dispensing duct (3) being horizontally partitioned—by a partitioning septum (4)—into an upper duct (3a) which dispenses said primary air flow (FP) and a lower duct (3b) which dispenses a powder-free secondary air flow (FS), the aspiration of the air forming the primary flow (FP) occurring through at least three air intakes (7) formed in the first portion (1) which are preferably arranged symmetrically with respect to the longitudinal centreline plane of the inhaler, the aspiration of the air forming the secondary flow (FS) occurring through an air intake (8) obtained at the distal end of said lower duct (3b),
the inhaler being characterised in that said base for supporting the cartridge (C) includes a plurality of horizontal support surfaces (9) projecting into the first portion (1) and oriented flow channels (11) formed in the support base which extend between said at least three air intakes (7) and inner powder fall region (5).

2. The delivery device according to claim 1, wherein said first portion (1) and said second portion (2) consist of the same polymeric material made of a single piece, said polymeric material being preferably selected from polyethylene (PE), low-density polyethylene (LORE), medium-density polyethylene (MORE), high-density polyethylene (HOPE), polypropylene (PP), polyvinyl acetate (PVC) and polycarbonate (PC).

3. The delivery device according to claim 2, wherein, in said dry-powder inhaler, a connection between the inner region (5) for the fall of the powder and the upper duct (3a) is obtained by means of a grid (6), and wherein said grid (6) consists of a single piece made of the same polymeric material as the first portion (1) and the second portion (2).

4. The delivery device according to claim 1, wherein said cartridge (C) comprises a cartridge wall circumscribing a compartment for containing said dry powder composition for inhalation wherein said cartridge wall comprises at least one moisture barrier layer, preferably a metal moisture barrier layer, so that a moisture content of said powder is comprised in the range of from 0.01% to 15% by weight with respect to the total weight of the powder, preferably from 0.1% to 10%, more preferably from 0.5% to 5%, for a predefined period of time

5. The delivery device according to claim 1, wherein said mixture (M) comprises or, alternatively, consists of acetylsalicylic add or a salt thereof and at least one first carrier, wherein said at least one first carrier is selected from the group comprising or, alternatively, consisting of: lactose, mannose, a hyaluronic add or a pharmaceutically acceptable salt thereof, preferably sodium hyaluronate, a dextran, and mixtures thereof.

6. The delivery device according to claim 1, wherein said powder composition or said mixture (M) has a volume median geometric diameter (VMGD) comprised in the range from 1 μm to 50 μm, preferably from 1 μm to 20 μm, more preferably from 1 μm to 5 μm.

7. The delivery device according to claim 1, wherein said dry powder composition comprises acetylsalicylic add or a salt thereof in a percentage by weight comprised in the range from 10% to 90% with respect to the total weight of the composition; preferably from 20% to 80%; more preferably from 30% to 70%.

8. The delivery device according to claim 1, wherein said inhaler is characterised in that the partitioning septum (4) is shorter than the dispensing duct (3), preferably by a distance (d) comprised between 4 and 7 mm.

9. The delivery device according to claim 1, wherein said inhaler is characterised in that the air intake (8) of the lower duct (3b) is partitioned into a plurality of air inlet regions, preferably having the same width, by one or more elongated septa (15), preferably not more than seven, extending below the powder fall region (5).

10. The delivery device according to claim 1, wherein said inhaler is characterised in that the first portion (1) comprises a perimeter wall (12) extending above the support base and it is provided with coupling means for locking the cartridge (C) onto the delivery device.

11. The delivery device according to claim 1, wherein said inhaler is a single-dose disposable inhaler; preferably wherein the cartridge (C) comprising the dry powder composition is fixed in the inhaler support base.

12. The delivery device according to claim 1, wherein said dry powder composition for inhalation is a composition for use in the curative or preventive treatment of thrombohemolytic events.

13. The delivery device according to claim 1, wherein said dry powder composition for inhalation is a composition for use in the curative or preventive treatment of thrombohemolytic events selected from: transient ischemic event, stroke, myocardial infarction.

14. A method comprising administering acetylsalicylic add or a salt thereof to a patient using a device of claim 1.
